(19) United States
(12) Patent Application Publication
(10) Pub. No.: US 2008/0206094 A1
(43) Pub. Date: Aug. 28, 2008

(54) Method and Apparatus for Collection and Treatment of Encapsulated Waste

(76) Inventor: Clifford C. Holloway, Willage (AU)

Correspondence Address: NIXON & VANDERHYE, PC, 901 NORTH GLEBE ROAD, 11TH FLOOR, ARLINGTON, VA, 22203, US

(21) Appl. No.: 11/573,585

(22) PCT Filed: Aug. 9, 2005
(86) PCT No.: PCT/AU05/01185

§ 371 Date: Feb. 12, 2007

(30) Foreign Application Priority Data
Aug. 11, 2004 (AU) 2004904499

(51) Int. Cl. A61L 2/025; B01J 3/04; B06B 1/00; A61L 2/07

(52) U.S. Cl. 422/20; 422/295; 422/128; 422/26

(57) ABSTRACT

An autoclave reactor for treating municipal solid waste (MSW) including agricultural waste and medical waste wherein the MSW may be encapsulated in plastic bags. The reactor is equipped with a loading internal guiding means for treating the waste in such a manner as to decompose the organic fraction into a pulp which acts as a fluid whereby rigid, material such as metal and glass may be removed by known means. The reactor may also be equipped with: (a) piercing and cutting devices for rupturing the encapsulating material thereby expelling air and releasing the contents, (b) means for collecting vapours and emissions for treatment before they are released into the environment, (c) a vacuum means for removing vapours and drying the resulting products, (d) a sensing means which determines the viscosity of the reactor contents and this information is fed into a computer which determines when the desirable decomposition has been reached.

### FIELD OF INVENTION

The present invention relates generally to the art of collection and disposal of waste products. It has particular application to the collection, treatment, and recycling of encapsulated waste, such as municipal solid waste (MSW) including medical waste and agricultural waste.

### BACKGROUND ART

For the purpose of this specification, waste products refer to household waste ‘garbage’, which is encapsulated in garbage bags, usually plastic bags, and medical waste encapsulated in bags and rigid containers, MSW.

It is common in certain fields to enclose waste in sealed containers such as plastic bags, to facilitate removal for disposal. One particular instance of such packaging is in the case of medical waste wherein the waste is packaged, not only for convenience of handling but also to ensure that pathogens are not spread inadvertently.

Alternative means of treatment of MSW have previously been disclosed by the present applicant in a series of patents, amongst which are issued in the U.S. Pat. No. 4,342,830 dated 3 Aug. 1982. U.S. Pat. No. 4,540,495 dated 10 Jun. 1985. U.S. Pat. No. 4,844,351 dated 4 Jul. 1989. U.S. Pat. No. 4,190,226 dated 2 Mar. 1993. U.S. Pat. No. 5,361,994, dated 8 Nov. 1994. U.S. Pat. No. 5,427,650 dated 27 Jun. 1995. Each of these have been primarily concerned with providing apparatus and method to improve the amount of waste that may be recycled. However each patent discloses a treatment of the waste by steam and pressure in a manner which sterilizes the waste. In addition the methods include a means of thorough agitation and mixing which ensures that all of the waste is effected by the steam, heat and pressure. While the process defined in the applicant's patents mentioned above have been found to effectively sterilize the waste, even medical waste, they have nonetheless been found to be inefficient in treating waste encapsulated in multiple bags and rigid containers. The treatment process requires substantial processing time and energy to break down the bags and other containers sufficiently to ensure thorough agitation and sterilization of the contents. This requires a considerable energy input beyond what would normally be required to treat the waste, therefore the process is inefficient. The waste included in the bag is small compared to the volume of the sealed bag, because of the volume of air in the bag. If a pressure vessel, as disclosed in the applicants patents mentioned above is filled with bagged waste, the quantity of waste held is quite small compared with the volume of the pressure vessel. The treatment process requires a substantial processing time to break down the plastic of the waste bags to sufficiently ensure thorough agitation and sterilization of the contents. This requires a considerable energy input just to remove the waste from its encapsulation so that it may be exposed to treatment.

The proceeding discussion of the background to the invention is intended only to facilitate an understanding of the present invention. It should be appreciated that the discussion is not an acknowledgment or admission that any of the material referred to was common knowledge in Australia at the priority date of this application.

### DISCLOSURE OF THE INVENTION

Accordingly, the invention resides in a waste collection and processing unit adapted to process encapsulated waste. The process unit comprising an autoclave ratable about an axis, drive means adapted to cause rotation the autoclave, defining a chamber with an inlet, a first closure adapted to sealing close the inlet, guide means within the chamber adapted to coax the waste away from the inlet during the rotation of the autoclave, piercing means adapted to pierce the capsules on contact and a port adapted to be connected to a source of sterilizing medium.

According to a preferred feature of the invention, the chamber is further provided with an outlet and and a second closure adapted to sealingly close the outlet, the guide means being adapted to coax the waste to move from the inlet to the outlet during the rotation of the pressure vessel.

According to a preferred embodiment, the sterilizing medium is steam.

According to a preferred feature of the invention, the piercing means is provided in a portion of the autoclave proximate to the inlet.

According to a preferred embodiment, the autoclave is substantially cylindrical, the chamber thereby defined having an inner surface, and the guide means comprises a helical rigid strip extending substantially perpendicularly inwardly from the inner surface into the chamber. (flighting)

According to a preferred embodiment, the axis about which the autoclave rotates is inclined to the horizontal, the inlet being positioned at a level above the outlet.

According to a preferred embodiment the guide means is configured helical to extend substantially along the length of the inner surface of the cylindrical pressure vessel.

According to a preferred embodiment the guide means may be of variable dept. into the cylinder in order to control the flow of the material.

According to a preferred embodiment, the pitch of the helical guide means reduces proximate to the outlet.

According to a preferred feature of the invention, the piercing means is associated with the guide.

According to a preferred embodiment, the piercing means comprises knife members releasably supported by the guide means.

According to a preferred embodiment, the knife members extend from the inner edge of the guide means inwardly into the chamber.

According to a preferred embodiment, the piercing means comprises serrations formed on the guide means.

According to a preferred feature of the invention, the process unit further comprises loading means associated with the inlet.

According to a preferred feature of the invention, an air inlet is provided proximate to the inlet, the air inlet being adapted to be connected to a source of pressurized air, which may be heated.

According to a preferred feature of the invention the autoclave is provided with a vacuum inlet, the vacuum inlet being adapted to be connected to an evacuating means.

According to a preferred feature of the invention, a water inlet is provided proximent to the inlet, the water inlet adapted to be connected to a source of pressurized water or other fluids.

According to a preferred feature of the invention, the pressure vessel is associated with a barrier which extends around the around the exterior of the pressure vessel and isolates the inlet from the outlet.

According to a preferred feature of the invention, the inlet is located in an enclosure accommodating the loading means, the enclosure isolating the inlet from the ambient surroundings.

According to a preferred embodiment, the barrier comprises a wall of the enclosure.

According to a preferred feature of the invention, the process unit further comprises a collecting zone associated with the outlet which is isolated from the ambient surroundings.

According to a preferred feature of the invention, the process unit further comprises a sensing means adapted to sense the viscosity of the fluidised waste, the sensing means being adapted to provide a signal when a predetermined level of viscosity of the fluidised waste has been attained.

According to a preferred feature of the invention, the process unit is microprocessor controlled.

According to a preferred feature of the invention, the process unit may be mounted on a truck or other mobile means.

According to a preferred feature of the invention, the mobile process unit may be used as a collection compactor (garbage compactor truck}.

The invention will be more fully understood in light of the following description of several specific embodiments.

### BRIEF DESCRIPTION OF THE DRAWINGS

The description is made with reference to the accompanying drawings of which:

FIG. 1 is a diagrammatic side elevation of a waste process autoclave unit according to a first embodiment;

FIG. 2 is a another diagrammatic side elevation of a first embodiment;

FIG. 3 is a further diagrammatic side elevation of the first embodiment;

FIG. 4 is a side elevation of the cylindrical vessel of the first embodiment having the near wall cut-away;

FIG. 5 is a partial sectional view of the cylindrical vessel of the first embodiment through section A-A shown in FIG. 4;

FIG. 6 is a side view of flighting as shown in FIG. 5;

FIG. 7 is a partial sectional view of the cylindrical vessel through line A-A shown in FIG. 4 according to a second embodiment;

FIG. 8 is a side view of flighting as shown in FIG. 7;

FIG. 9 is side view of flighting as arranged in FIG. 7 according to a third embodiment:

FIG. 10 is a view of the autoclave unit mounted on a truck;

FIG. 11 is a longitudinal diagrammatic section showing alternative positioning of the helical guide allowing for a kneading action and mastification of the organics (chicken craw)

### BEST MODE(S) FOR CARRYING OUT THE INVENTION

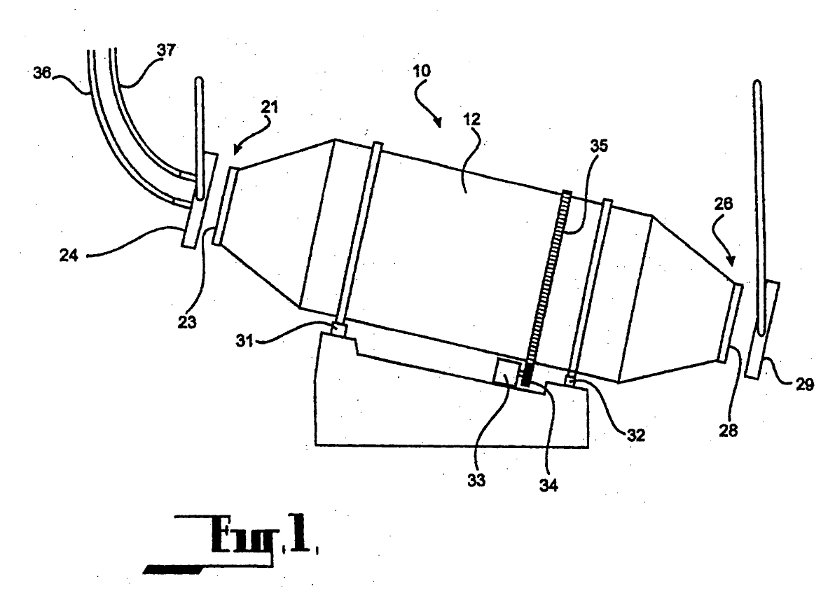

The first embodiment of the invention as shown at FIGS. 1, 2 and 3 comprises a process unit 10 adapted to process waste (including medical waste) encapsulated in plastic and other containers. The process unit comprises a substantially cylindrical autoclave 12 which is supported by support members 31 and 32 which may be mounted on a mobile vehicle or may be stationary. Support members provide for rotation about its longitudinal axis. The cylindrical vessel 12 is adapted to be caused to rotate by means of a motor 33 having an output gear 34 adapted to engage a gear 36 secured around the periphery of vessel 12. The cylindrical vessel 12 is inclined at a predetermined angle to facilitate the movement waste through the cylindrical vessel 12.

The cylindrical vessel 12 defines an internal chamber 50 having an inlet 23 at one end 21 of the cylindrical vessel 12 and an outlet 28 at the other end 26. The process unit 10 is further provided with a first closure adapted to close the inlet 23 and a second closure 29 adapted to close the outlet 28. Each of the closures are supported from above the cylindrical vessel 12 by support means adapted to move the closure from a position wherein it is engaged with the respective end to a position upwardly clear of the respective end when disengaged from the respective end.

The inlet closure is provided with a first aperture (not shown) which is connected to a source of steam through a steam line 36. The inlet closure is further provided with a second aperture (not shown) which is connected to a source of compressed air through an air line 37. The inlet closure is further provided with a third aperture (not shown) which is connected to a vacuum pump through a vacuum line 44.

A first enclosure 42 is associated with the inlet to substantially contain the inlet and to collect any air expelled from the chamber when waste is delivered to it. Air from within the first enclosure 42 is exhausted through an air treatment means adapted to destroying any pathogens which may be present in the air prior to the air being exhausted into the atmosphere.

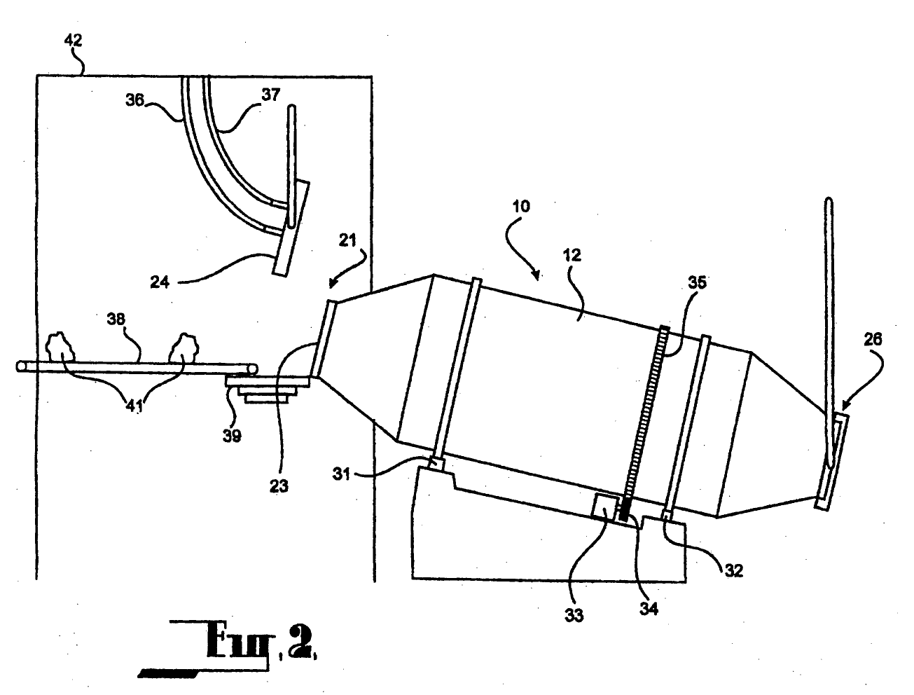

The process unit 10 further comprises an inlet conveyor 38 and a sub-conveyor 39 (see FIG. 2) adapted to deliver bagged waste to the inlet. The sub-conveyor is adapted to be movable away from the inlet to enable the inlet to be closed by the first closure 24. The conveyor 38 and sub-conveyor 39 penetrate the first enclosure to transport bagged waste to the cylindrical vessel 12.

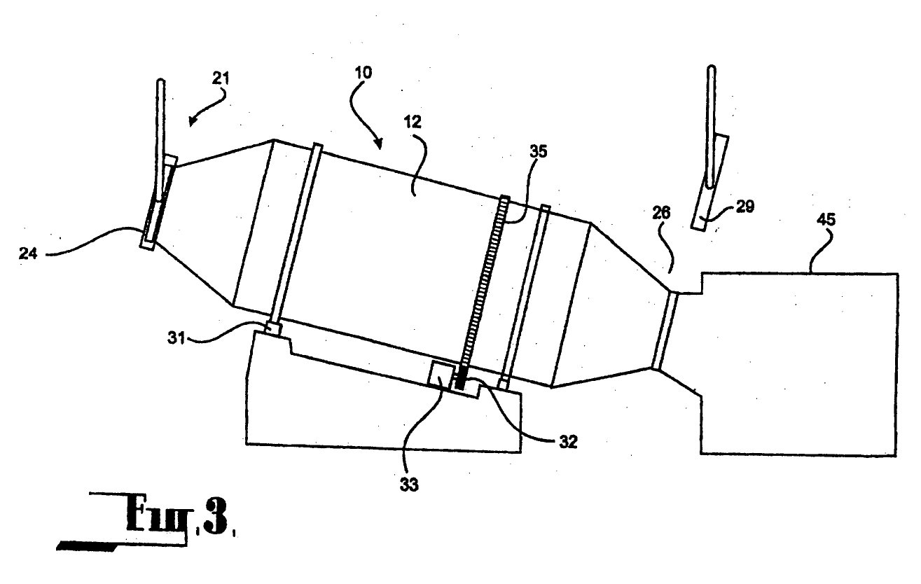

As shown in FIG. 3, the process unit may be connected to a waste collection and sorting means 45 adapted to receive processed waste from the outlet after completion of the process cycle. In addition the collecting and sorting means comprises means to classify the treated waste into predetermined classes. In general, suitable collection and sorting means have been described in the the applicant's previous US patents mentioned in the description of the background to this invention, above.

In addition, the collection and sorting means is provided with a filtration means to collect airborne waste expelled from the cylindrical vessel by compressed air. The filtration means comprises a mesh filter exhausting to the environment at the end of a duct directing the airborne waste away from the path of the more dense waste.

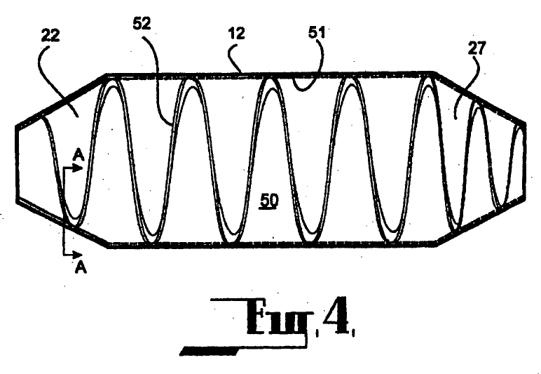

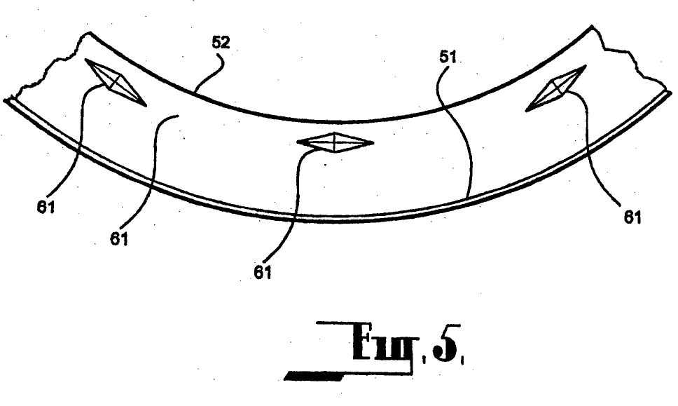

As shown in FIG. 4, the cylindrical vessel has an internal surface 51 to which is attached flighting 52. The flighting 52 comprises a rigid strip extending substantially perpendicularly inwardly into the chamber 50 from the inner surface 51. The flighting 52 is configured helically too extend substantially along the full length of the inner surface 51 of the cylindrical vessel. A sparging steam line may be incorporated into the flighting as described in US patents above. This steam line may be connected to piercing members 61 FIG. 5, so that steam is injected into the bags as they are being pierced

The pitch of the helix of the flighting is reduced in the outlet portion 27, producing compression on the waste. For ease of maintenance, the flighting 52 may be constructed from a series of segments configured end to end arrangement and reasonably secured to the inner surface 51.

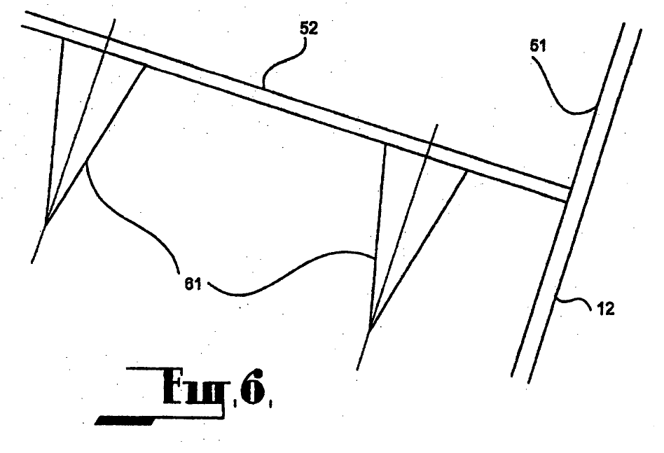

In the region of the chamber adjacent to inlet 23 and as shown in more detail in FIGS. 5 and 6 The flighting 52 is provided with piercing means in the form of a series of knife members 61 projecting from the flighting 52. each knife member 61 comprises a rigid blade having a substantially diamond cross-section to provide stiffness and further comprises a relatively broad end engaging the flighting, or sparging line and a substantially pointed opposed end projecting into the chamber 50. The broad end and the pointed end are connected by edges of the blade which are sharpened. The knife members are sharpened. the knife members are releasably attached to the flighting or sparging line (not shown) to enable replacement in the event of damage or breakage to the knife members 61.

In use, bagged waste 41 is loaded into the cylindrical vessel 12 through inlet 23 by the conveyor 38 while the cylindrical vessel 12 is rotating, outlet 28 being closed by second closure 29. After entry into chamber 50, the container (bag) 41 is moved about within the chamber 50 because of the rotation of the cylindrical vessel 12. When a bag 41 contacts the pointed end of a knife member 61 in the inlet portion of chamber 50, the bag 41 is pierced. The rotational movement of the cylindrical vessel 12 ensured that the bag (container) 41 is torn open after it is pierced and injected by steam, thereby releasing and spreading the contents within chamber 50. Further as a result of the rotational motion the flighting urges the waste away from the inlet, toward the outlet portion. As a result the waste is pre-compacted during the loading process, increasing the vessel capacity by 50% or more. Loading continues until sufficient waste has been inserted. Thereafter the inlet is closed by the first closure 24 and steam under pressure is introduced into chamber 50. The cylindrical vessel 12 is rotated and pressurized steam is directed into the chamber 50 through the steam line 54, expelling air through a relief valve (not shown) and filling the cylindrical vessel 12 with steam under pressure. The helical configuration of the flighting 52 transports the waste materials to the closed end of the vessel. Since the exit closure member 16 is closed and there is no exit, the waste material is squeezed back (extruded) through that portion of the chamber between the flighting in a manner previously described in the applicant's patent no. U.S. Pat. No. 5,190,446. This process causes any paper (cellulose) to be torn apart by the compression and sheer action of this indirect extrusion.

After a predetermined viscosity or period of time which depends on the nature of the waste being being processed, the pressure within the vessel is released. The steam remaining in the autoclave is evacuated through the vacuum line 44. A predetermined vacuum depending on the nature of the waste being treated, causes the the moisture content of the waste to be reduced. As a result of the heat and vacuum the cellulose material present takes on a finely shredded, fluffy appearance as a result of being exposed to the vacuum and this material is easily able to be displaced by a blast of air. Upon completion of the evacuation phase, air is admitted into the autoclave to bring the priddure to atmospheric pressure. The second closure member 29 is opened. The cellulose material is expelled by directing a draft of pressurized air from the air line 37 into the chamber 50. This airborne waste is collected by the filtration means to collect airborne waste within the waste collection and sorting means 45. The remainder of the processed waste which is solid or paste like is forced out of the chamber 50 by the rotary of the flighting 52.

In a preferred adaptation of the first embodiment, the inlet closure is provided with a third aperture (not shown) which is connected to a source of water by a water line. a spray nozzle is mounted to the inner side of the third aperture. In use, after the cylindrical vessel is emptied of the waste, water from the water line is sprayed into the chamber to clean the chamber of remaining refuse.

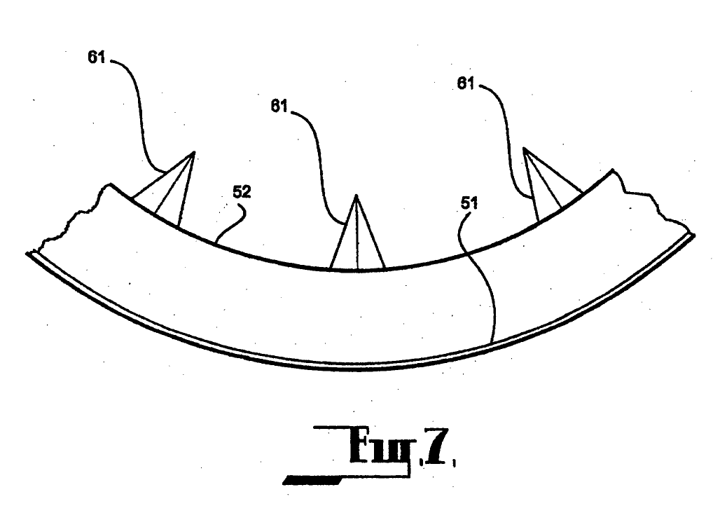

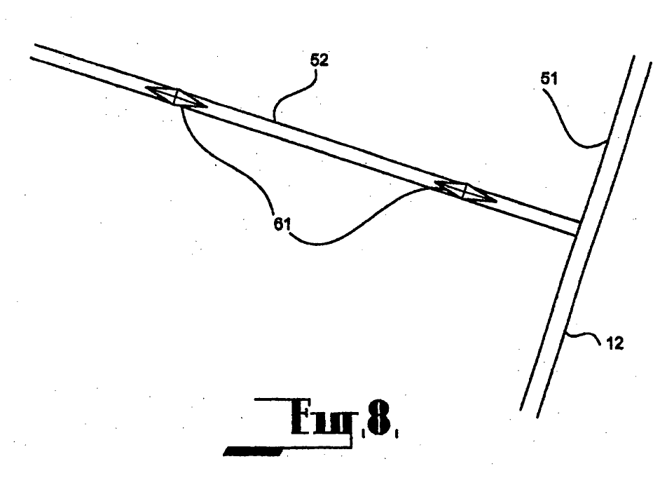

In a second embodiment, as shown in FIGS. 7 and 8, the knife members are mounted to the inner edge of the flighting 52 and extend radially inwardly from the inner edge into the chamber 50.

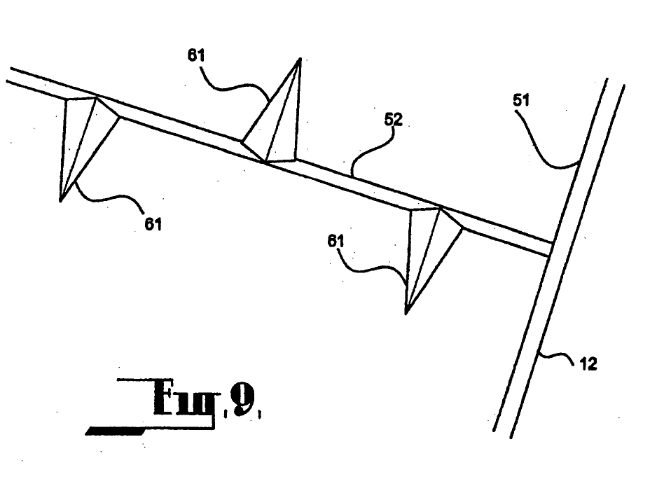

In a third embodiment as shown in FIG. 9, the knife members may be directed at obliquely to the radial direction.

In a fourth embodiment, not shown, the piercing means comprises saw tooth-like serration's, not shown, associated with the inner edge 52 of the flighting. The internal edge of flighting has saw-tooth like edging to the flighting.

In a fifth embodiment, the serrations are provided by a metal strip with the serrations preformed on one edge, the strip being adapted to be secured to the flighting proximate the edge of the edge of the flighting.

In a sixth embodiment, the pressure unit comprises a pressure vessel rotatable about an axis and having a motor adapted to cause rotation of the pressure vessel in a manner similar tooth described with respect to the first embodiment. The pressure vessel defines a chamber with an inlet and a first closure adapted to sealingly close the inlet. Guide means and piercing means are provided within the chamber, in a manner similar to that of the first or subsequent embodiments. However, in this embodiment, no separate outlet is provided.

In a further adaptation of any of the previous embodiments, a second enclosed space in the form of a room encloses the inlet end of the cylindrical vessel, the cylindrical vessel penetrating an opening in a wall of the room intermediate the ends of the cylindrical vessel. The conveyor, 38, the sub-conveyor 39 and first enclosure are contained within the second enclosure, A sealing means is associated with the opening of the wall, the sealing means being adapted to sealingly engage the cylindrical vessel without impeding rotation. In this manner, the outlet of the cylindrical vessel is isolated from the inlet. The room containing the inlet may then be treated as a hazardous area where in it is recognized that there is a risk of contact with pathogens and in which appropriate safety precautions should be taken. By operation of the process unit as described above, the outlet of the cylindrical vessel need never be opened while the inlet is open, other than for maintenance of the process unit. When the second closure is opened, the inlet is closed by the first closure and processing of the waste has been completed rendering the chamber of the cylindrical vessel and its contents are completely sterilized. In this way, the area adjacent to the outlet may be treated as a safe area with regards to the spread of pathogens wherein no special precautions are required.

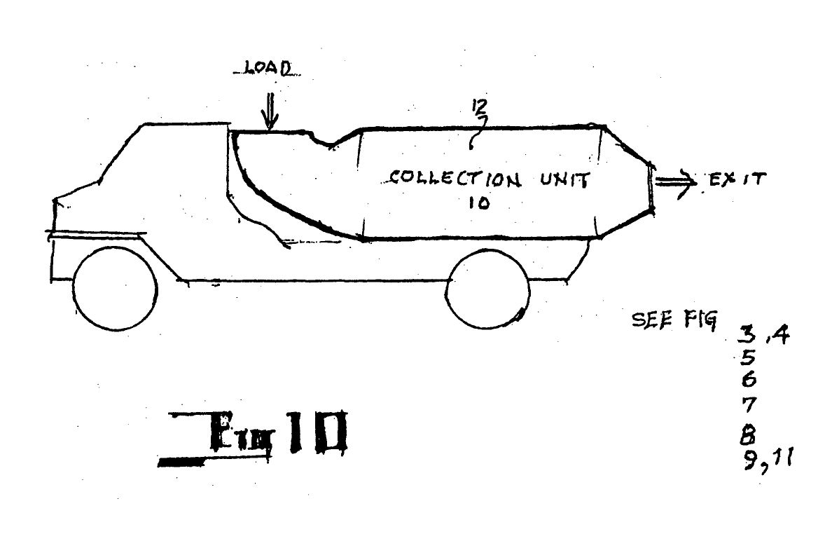

In this embodiment the loading means and the waste collection and sorting means are adapted so that they may be alternately positioned adjacent to the opening and otherwise moved clear of the opening, in other respect the ampetures for steam etc. are best located in the closed end. This embodiment may be adapted for mobile operation for waste collection, (waste compactor truck). See FIG. 10.

In use, when processing is complete, the fluffy cellulose waste is removed by directing a stream of air from the airline into the chamber. the cellulose waste is expelled through the opening as a result and the waste filtered in the manner of the first embodiment. The remaining waste is removed from the chamber by reversing the rotation of the pressure vessel, whereby it is coaxed by the flighting. In other respects, this embodiment may be be adapted as previously described in respect of the other embodiments.

In a further adaptation of any of the previous embodiments, the process unit further comprises a sensor adapted to detect the viscosity of the processing waste. The sensor is adapted to signal when when the processed waste has reached a predetermined level of fluidisation. This level of fluidisation more accurately identifies when the waste has been processed sufficiently. The signaling by the sensor may then be used as a means to identify when the processing should cease, rather than operating the process unit for specific time period.

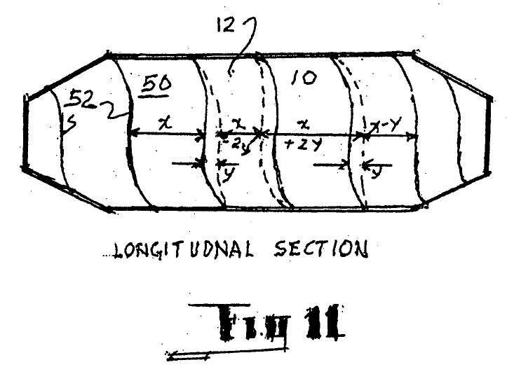

In an adaptation of the previous embodiments, the pitch spacing of the flighting (see FIG. 11) may be varied to produce a kneading effect on the material being processed. The spacing shown on FIG. 11 may be varied from x−2 to x+2 to produce this kneading. This compression-relaxation action, along with the broken glass and other abrasives inherent in MSW produces a mastification effect (chicken gizzard) resulting in particle size reduction for further fermentation or gasification.

In another adaptation of any of the previous embodiments, the process unit is microprocessor controlled. A microprocessor is connected to actuate the motor, 33, the closures 24 and 29, the vent fan the conveyor 38 and sub-conveyor 39, steam air and water valves and waste collection and sorting means 45. Automatic operation of the process unit is established in a pre-determined manner, with appropriate safety interlocks in place to ensure that the operation occurs only after appropriate precautions have been taken.

The process unit according to the above embodiments provides a means for processing of bagged waste in which a substantial quantity of bagged waste may be processed in an efficient and economic manner in a way in which the environmental impact is considerably reduced from previous methods, by use of this process unit, a process for processing bagged waste may be implemented which incorporate procedures for safe handling of bagged waste containing pathogens. The process is further adapted to the use of techniques previously identified for for the processing municipal solid waste, which substantially enhances the proportion of waste recovered for recycling. In addition, when used to process medical waste, the process is effective in separating a needle or other “sharps” from their plastic hypodermic syringe. Such needles are then able to be separated for re-cycling. As a result of this separation of the sharps, the need for hammer-mill is removed. This reduces operating costs considerably.

It should appreciated that the scope of the present invention need not be limited to the particular scope of the embodiments described above.

1-17. (canceled)

18. A waste processing unit for treating encapsulated municipal solid waste comprising:
an autoclave rotatable about an axis, the autoclave defining a chamber with an inlet;
a driver adapted to cause rotation of the autoclave;
a first closure adapted to sealingly close the inlet;
a guide within the chamber adapted to coax the waste away from the inlet during rotation of the autoclave; and
a piercing mechanism adapted to pierce the encapsulated waste.

19. A waste processing unit according to claim 18, wherein the unit further comprises a port adapted to be connected to a source of sterilizing medium.

20. A waste processing unit according to claim 18, wherein the chamber is further provided with an outlet and a second closure adapted to sealingly close the outlet, the guide means being adapted to coax the waste to move from the inlet to the outlet during the rotation of the pressure vessel.

21. A waste processing unit according to claim 19, wherein the sterilizing medium is steam.

22. A waste processing unit according to claim 18, wherein the piercing mechanism is provided in a portion of the autoclave proximate to the inlet.

23. A waste processing unit according to claim 18, wherein the autoclave is substantially cylindrical.

24. A waste processing unit according to claim 18, wherein the chamber has an inner surface and the guide comprises a helical rigid strip extending substantially perpendicularly inwardly from the inner surface of the chamber.

25. A waste processing unit according to claim 24, wherein the guide is configured helically to extend substantially along the length of the inner surface of the chamber.

26. A waste processing unit according to claim 25, wherein the pitch of the helical guide reduces proximate to the outlet.

27. A waste processing unit according to claim 25, wherein the pitch spacing of the helical guide is varied to produce a kneading effect on the waste being processed.

28. A waste processing unit according to claim 27, wherein the pitch spacing is varied from (x−2) to (x+2) to produce the kneading effect.

29. A waste processing unit according to claim 18, wherein the axis about which the autoclave rotates is inclined to the horizontal, the inlet being positioned at a level above the outlet.

30. A waste processing unit according to claim 18, wherein the guide is of variable depth into the cylinder in order to control the flow of the material.

31. A waste processing unit according to claim 18, wherein the piercing mechanism is associated with the guide.

32. A waste processing unit according to claim 31, wherein the piercing mechanism comprises knife members releasably supported by the guide.

33. A waste processing unit according to claim 32, wherein the knife members extend from the inner edge of the guide inwardly into the chamber.

34. A waste processing unit according to claim 31, wherein the piercing mechanism comprises serrations formed on the guide.

35. A waste processing unit according to claim 34, wherein the serrations are provided by a metal strip with the serrations preformed on one edge of the strip, the strip being adapted to be secured to the guide proximate the edge of the guide.

36. A waste processing unit according to claim 18, wherein the unit further comprises a loader associated with the inlet.

37. A waste processing unit according to claim 18, wherein an air inlet is provided proximate to the inlet, the air inlet being adapted to be connected to a source of pressurized air, which may be heated.

38. A waste processing unit according to claim 18, wherein the autoclave is provided with a vacuum inlet, the vacuum inlet being adapted to be connected to suction device.

39. A waste processing unit according to claim 38, wherein air and gases are evacuated through the suction device.

40. A waste processing unit according to claim 39, wherein the air and gases passing through the suction device are exhausted through an air treatment means adapted to destroy any organic molecules and pathogens in the air or gas.

41. A waste processing unit according to claim 18, wherein a water inlet is provided proximate to the inlet, the water inlet adapted to be connected to a source of pressurized water or other fluids.

42. A waste processing unit according to claim 18, further comprising a sensor adapted to sense the viscosity of the fluidized waste, the sensor being adapted to provide a signal when a predetermined level of viscosity of the fluidized waste has been attained.

43. A waste processing unit according to claim 18, wherein the unit is microprocessor controlled.

44. A waste processing unit according to claim 18, wherein the processing unit is mounted on a transporter.

45. A waste processing unit according to claim 44, wherein exhaust air from the transporter is diverted to the chamber of the autoclave.

46. A waste processing unit according to claim 45, wherein the exhaust air is compressed, heated air.

47. A waste processing unit according to claim 45, wherein the exhaust air includes carbon monoxide and other compounds and molecules such as free radicals.

48. A waste processing unit according to claim 18, further comprising a microcomputer which determines when desirable decomposition has been reached thereafter stopping the operation.

49. A waste processing unit according to claim 18, wherein agricultural waste is added to the municipal solid waste and an acid in the agricultural waste decomposes cellulostic material in the municipal solid waste.

50. A waste processing unit according to claim 18, wherein agricultural waste is added to the municipal solid waste, wherein broken glass in the municipal solid waste combined with the compression/relaxation action of the autoclave produces a mastication effect on the agricultural waste reducing the particle size and exposing cellulose in the agricultural waste to reaction forces to increase the desirable lignocellulose content and dispose of a by-product of sugar production.

51. A waste processing unit according to claim 27, wherein steam, heat and pressure together with a kneading effect of the guide decomposes organic matter in the municipal solid waste, and the decomposed organic matter acts as a fluid, and crystalline material is aligned and subject to compression and relaxation activity resulting in a piezoelectric effect thereby further decomposing the organic material.

52. A waste processing unit according to claim 18, wherein steam, heat, pressure and mechanical forces acting on a fluidised pulp formed in the autoclave produces cavitation activity which is increased by adding ultrasonic devices.

53. A waste processing method for treating encapsulated municipal solid waste, the method comprising:
rotating an autoclave about an axis, the autoclave defining a chamber with an inlet;
sealing the inlet after placing the waste in the chamber;
pressurizing the chamber after sealing the inlet;
coaxing the waste away from the inlet during rotation of the autoclave with a guide in the chamber; and
piercing the waste encapsulated in the chamber.

54. A method as in claim 53, comprising injecting a sterilizing medium into the chamber after sealing the inlet.

55. A method as in claim 53 wherein coaxing the waste includes moving the waste from the inlet to an outlet of the chamber during the rotation of the chamber.

56. A method as in claim 54, wherein the sterilizing medium is steam.

57. A method as in claim 53 wherein the guide is a helical rigid strip extending substantially perpendicularly inwardly from the inner surface of the chamber and coaxing includes moving the waste along the helical rigid strip.

58. A method as in claim 53 further comprising pressurizing the chamber with heated pressurized air.

59. A method as in claim 53 further comprising evacuating the chamber of air and gases.

60. A method as in claim 53 further comprising sensing a viscosity of fluidized waste in the chamber and generating a signal when a predetermined level of viscosity of the fluidized waste is attained.

61. A method as in claim 53 further comprising introducing exhaust air from an internal combustion engine to the chamber.

62. A method as in claim 53 further comprising adding agricultural waste to the municipal solid waste in the chamber and using the agricultural waste to decompose cellulostic material in the municipal solid waste.

63. A method as in claim 62 further comprising using broken glass in the municipal solid waste and a compression and relaxation action of the autoclave chamber rotation to produce a mastication effect on the agricultural waste which reduces particle size and exposes cellulose in the agricultural waste to reaction forces to increase the desirable lignocellulose content and dispose of a by-product of sugar production.

64. A method as in claim 53 further comprising adding pressurized steam to the chamber to facilitate decomposition of organic matter in the municipal solid waste; forming a fluid from the decomposed organic matter acts, and the rotation of the chamber applies a compression and relaxation to crystalline material in the solid waste to produce a piezoelectric effect which further decomposing the organic matter.

65. A method as in claim 53 further comprising applying ultrasonic energy to the assist in cavitating fluidized waste in the chamber.
